(19) United States
(12) Patent Application Publication
(10) Pub. No.: US 2026/0158245 A1
(43) Pub. Date: Jun. 11, 2026

(54) BALLOON CATHETER INCLUDING TENSIONING MEMBER

(71) Applicant: BOSTON SCIENTIFIC SCIMED, INC., MAPLE GROVE, MN (US)

(72) Inventors: Adam David Grovender, Maple Grove, MN (US); James M. Anderson, Corcoran, MN (US); Benjamin Jerome Bidne, Loretto, MN (US)

(73) Assignee: BOSTON SCIENTIFIC SCIMED, INC., MAPLE GROVE, MN (US)

(21) Appl. No.: 19/179,348

(22) Filed: Apr. 15, 2025

Related U.S. Application Data
(60) Provisional application No. 63/634,601, filed on Apr. 16, 2024.

(51) Int. Cl. A61M 25/10; A61F 2/24

(52) U.S. Cl. CPC A61M 25/1002; A61M 25/104; A61F 2/2433; A61M 2025/1004; A61M 2025/1068; A61M 2205/581; A61M 2205/582

(57) ABSTRACT

Example medical devices and methods of use are disclosed. An example medical device includes a balloon catheter including a balloon having a proximal waist, a distal waist and a body portion extending therebetween. The medical device also includes an outer elongate member having a proximal end region, a distal end region and a lumen extending therein. The medical device may also include an inner elongate member extending within a portion of the lumen of the outer elongate member. The medical device may also include a manifold coupled to the proximal end region of the outer elongate member and may further include a tensioning member coupled to the proximal end region of the inner elongate member. The tensioning member is configured such that translating the tensioning member relative to the manifold allows axial tension to be maintained on the inner elongate member during deflation of the balloon.

### CROSS-REFERENCE TO RELATED APPLICATION

This application is a continuation of U.S. Patent Application Ser. No. 63/634,601, filed Apr. 16, 2024, entitled “BALLOON CATHETER INCLUDING TENSIONING MEMBER”, which is incorporated by reference herein in its entirety.

### TECHNICAL FIELD

The present disclosure relates to methods and apparatus for performing valvuloplasty. More particularly, the present disclosure relates to methods and apparatus for performing valvuloplasty using an inflatable balloon catheter.

### BACKGROUND

Heart valve stenosis or calcification is a common manifestation in valvular heart disease, and may often be a leading indicator for balloon valvuloplasty and/or valve replacement therapy. In some instances, balloon valvuloplasty may be beneficial in improving the lifestyle of patients suffering from valve stenosis and may also contribute to a successful valve replacement procedure.

Stenotic or narrowed heart valves may be treated with a number of relatively non-invasive medical procedures including percutaneous transluminal balloon valvuloplasty (PTBV), percutaneous transcatheter heart valve replacement (PTVR) and combinations thereof. Valvuloplasty techniques typically involve advancing a balloon catheter over a guidewire and through an introducer sheath (e.g., an expandable introducer sheath), whereby the valvuloplasty balloon of the balloon catheter is positioned within the heart valve and inflated to dilate the narrowed heart valve.

In other examples, percutaneous transcatheter heart valve replacement (PTVR) may be performed to replace a diseased, native heart valve with an artificial heart valve. One method of performing percutaneous transcatheter heart valve replacement may include the use of a valvuloplasty balloon to dilate the stenotic heart valve prior to implantation of the heart valve.

In yet other examples, a valvuloplasty balloon may be utilized to expand a replacement heart valve placed within a stenotic heart valve. Accordingly, there is an ongoing and unmet need for improved valvuloplasty balloons and improved methods of treating valvular heart disease.

### BRIEF SUMMARY

This disclosure provides design, material, manufacturing method, and use alternatives for medical devices. An example medical device includes a catheter which includes a balloon having a proximal waist, a distal waist and a body portion extending therebetween. The catheter may further include an outer elongate member having a proximal end region, a distal end region and a lumen extending therein, the distal end region may further be coupled to the proximal waist of the balloon. The catheter may further include an inner elongate member extending within a portion of the lumen of the outer elongate member and the body of the balloon, in which a distal end region of the inner elongate member may be coupled to the distal waist of the balloon. The catheter may further include a manifold and the manifold may be coupled to the proximal end region of the outer elongate member. The catheter may further include a tensioning member coupled to the proximal end region of the inner elongate member, such that translating the tensioning member relative to the manifold may maintain axial tension on the inner elongate member during deflation of the balloon.

Alternatively or additionally to any of the examples above, the manifold may include a first locking region and the tensioning member may include a second locking region configured to engage the first locking region.

Alternatively or additionally to any of the examples above, the first locking region may be engaged with the second locking region such that axial tension is maintained on the inner elongate member, the balloon, or both the inner elongate member and the balloon during deflation of the balloon.

Alternatively or additionally to any of the examples above, the first locking region may interlock with the second locking region.

Alternatively or additionally to any of the examples above, the first locking region may irreversibly interlock with the second locking region.

Alternatively or additionally to any of the examples above, the first locking region may engage the second locking region via an annular snap-fit connection.

Alternatively or additionally to any of the examples above, the first locking region may interlock with the second locking region and provide tactile feedback, audible feedback or both tactile and audible feedback to a user upon interlocking.

Alternatively or additionally to any of the examples above, the first locking region may include a first plurality of threads and the second locking region may include a second plurality of threads configured to threadedly engage the first plurality of threads.

Alternatively or additionally to any of the examples above, the tensioning mechanism may be spaced away from the manifold in a first configuration, and the tensioning member may be translated toward the manifold to impart axial tension on the inner elongate member, the balloon or both the inner elongate member and the balloon.

Alternatively or additionally to any of the examples above, the manifold may further include an aperture extending along a lateral side of the manifold, and at least a portion of the tensioning member may be configured to extend through the aperture, and translating the tensioning member along the aperture may impart axial tension on the inner elongate member, the balloon or both the inner elongate member and the balloon.

Alternatively or additionally to any of the examples above, translating the tensioning member relative to the manifold may be configured to impart an axial tension force on the inner elongate member, the balloon, or both the inner elongate member and the balloon in a range from 1 to 10 Newtons (N).

Alternatively or additionally to any of the examples above, a catheter is disclosed. The catheter of this and other examples may include a balloon having a proximal waist, a distal waist, and a body portion extending therebetween. The catheter of this and other examples may further include an outer elongate member having a proximal end region, a distal end region and a lumen extending therein, wherein the distal end region may be coupled to the proximal waist of the balloon. The catheter of this and other examples may further include an inner elongate member extending within a portion of the lumen of the outer elongate member and the body of the balloon. A distal end region of the inner elongate member may be coupled to the distal waist of the balloon. The catheter of this and other examples may further include a manifold coupled to the proximal end region of the outer elongate member. The catheter of this and other examples may further include a tensioning member coupled to the proximal end region of the inner elongate member. The tensioning member may be spaced away from the manifold in a first configuration, and the tensioning member may be configured to interlock with a portion of the manifold in a second configuration, such that translating the tensioning member from the first configuration to the second configuration imparts axial tension on the inner elongate member, the balloon or both the inner elongate member and the balloon during deflation of the balloon.

Alternatively or additionally to any of the examples above, the tensioning member may be configured to irreversibly interlock with the manifold.

Alternatively or additionally to any of the examples above, the tensioning member may be configured to interlock with the manifold via an annular snap-fit connection.

Alternatively or additionally to any of the examples above, wherein interlocking the tensioning member with the manifold provides tactile feedback, audible feedback or both tactile and audible feedback to a user.

Alternatively or additionally to any of the examples above, wherein the tensioning member is configured to reversibly interlock with the manifold.

Alternatively or additionally to any of the examples above, wherein a portion of the tensioning member includes a first plurality of threads and wherein a portion of the manifold includes a second plurality of threads configured to threadedly engage the first plurality of threads.

Alternatively or additionally to any of the examples above, wherein translating the tensioning member from the first configuration to the second configuration imparts an axial tension force on the inner elongate member, the balloon, or both the inner elongate member and the balloon in a range from 1 to 10 Newtons (N).

An example method of using a balloon catheter for treatment includes advancing a catheter into a body vessel of a patient, the catheter including: a balloon having a proximal waist, a distal waist and a body portion extending therebetween; an outer elongate member having a proximal end region, a distal end region and a lumen extending therein, wherein the distal end region is coupled to the proximal waist of the balloon; an inner elongate member extending within a portion of the lumen of the outer elongate member and the body of the balloon, wherein a distal end region of the inner elongate member is coupled to the distal waist of the balloon; a manifold coupled to the proximal end region of the outer elongate member; and a tensioning member coupled to the proximal end region of the inner elongate member. Example methods further contemplate translating the tensioning member relative to the manifold to impart axial tension on the inner elongate member and balloon; inflating the balloon within the vessel of the patient; and deflating the balloon while maintaining axial tension on the inner elongate member and the balloon.

The above summary of some examples is not intended to describe each disclosed example or every implementation of the present disclosure. The Figures and Detailed Description, which follow, more particularly exemplify these examples.

### BRIEF DESCRIPTION OF THE DRAWINGS

The disclosure may be more completely understood in consideration of the following detailed description in connection with the accompanying drawings, in which:

FIG. 1 illustrates a balloon catheter deployed within the heart of a patient.

FIG. 2 illustrates an exemplary balloon catheter of the present disclosure.

FIG. 3 is a cross-sectional view of the balloon catheter taken along line 3-3 of FIG. 2.

FIG. 4 is a detailed view of the distal end of a balloon catheter according to the present disclosure.

FIG. 5 is a side view of a balloon catheter in an inflated configuration.

FIG. 6 is a side view of the balloon catheter of FIG. 5 in a deflated configuration.

FIG. 7 is a cross-sectional view of the balloon catheter taken along line 7-7 of FIG. 6.

FIG. 8 illustrates the proximal end of a balloon catheter according to the present disclosure.

FIG. 9 illustrates the proximal end of a balloon catheter according to the present disclosure.

FIGS. 10-11 illustrate the actuation of a tensioning member of a balloon catheter according to the present disclosure.

FIG. 12 is a side view of a balloon catheter according to the present disclosure.

FIG. 13 is a side view of a balloon catheter according to the present disclosure.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

The following description should be read with reference to the drawings wherein like reference numerals indicate like elements throughout the several views. The detailed description and drawings illustrate examples of the claimed disclosure.

As discussed above, medical balloons may be utilized in a variety of medical treatments. For example, in a valvuloplasty procedure, a valvuloplasty balloon may be used to expand a diseased heart valve. In a percutaneous transcatheter heart valve replacement (PTVR), a valvuloplasty balloon may be used to replace a diseased, native heart valve with an artificial heart valve.

Valvuloplasty balloons may be introduced into a patient by passing through an expandable introducer sheath, through which a guidewire may be placed. The valvuloplasty balloon may then be delivered to a target site by tracking the balloon catheter over the guidewire to the target site. In some cases, the pathway to a target site may be tortuous and/or narrow. Upon reaching the site, the valvuloplasty balloon may be expanded by injecting a fluid into the interior of the balloon. Expanding the valvuloplasty balloon may radially expand the stenotic heart valve such that normal blood flow may be restored through the valve.

In some instances, it may be desirable to utilize high pressure valvuloplasty balloons when treating a particular target site (e.g., a stenotic heart valve). To achieve the desired pressure or force against tissue at the target site, a valvuloplasty balloon may be constructed with a relatively large diameter and/or a thicker balloon wall. However, a larger and/or thicker wall may increase the folded profile (e.g., outer diameter) of the balloon when in a deflated configuration. Minimizing the profile of the balloon in a deflated configuration is important as the profile effects the ease and ability of the valvuloplasty balloon to pass through an introducer sheath, through the coronary arteries and across a narrowed heart valve. Further, a reduced re-folded balloon profile allows the deflated balloon to pass back through the introducer sheath with reduced withdrawal forces when the catheter is removed from the patient. To minimize the outer diameter of the balloon in its deflated condition, it may be desirable to control the folding/re-folding mechanics of the valvuloplasty balloon by applying tension to one or more components of the balloon catheter during deflation of the balloon. Examples disclosed herein may include balloon catheters designed to control the folding mechanics of the balloon by applying tension to one or more components of the balloon catheter during deflation of the balloon.

The term “flexural modulus” generally refers to an intensive property that is computed as the ratio of stress to strain in flexural deformation, or the tendency for a material to resist bending.

The term “tension” generally refers to force transmitted through an object or tether (i.e., a rope, string, cable, or similar object) to convey and apply that same magnitude of force to an element or device connected to the object or tether.

The term “motive force” generally refers to the conjunction term referring to the force or forces that cause something to move or, in a sense, a force that induces motor action.

The term “patient,” as used herein, comprises any and all organisms and includes the term “subject.” A patient can be a human or an animal.

The term “waist” as in “balloon waist” or “proximal balloon waist” or “distal balloon waist” generally refers to an inflection point wherein the diameter of the balloon abruptly changes or marks the transition into a change of diameter.

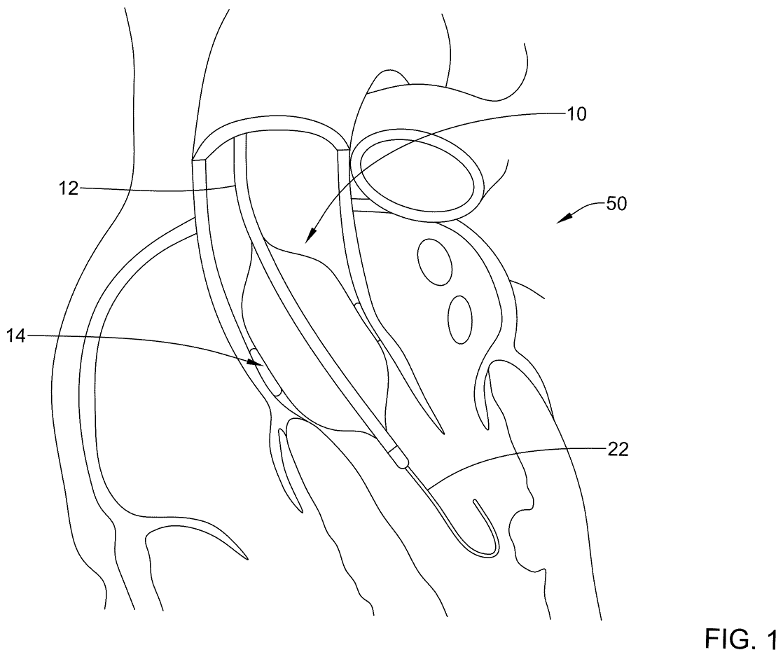

FIG. 1 illustrates an example balloon catheter 10 positioned in the heart 50 of a patient. As depicted in FIG. 1, the catheter 10 may be initially inserted through the lumen of an introducer sheath and tracked over a guidewire to a target treatment site (e.g., heart of a patient). Guidewire 22 may pass through a guidewire port and through the guidewire lumen of the catheter 10 to guide catheter 10 through a patient's vasculature.

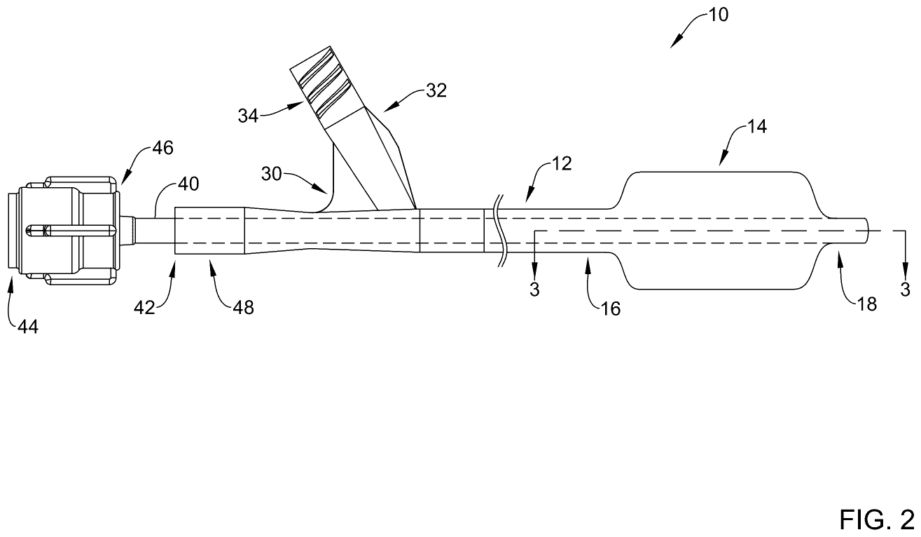

The balloon catheter 10 may include an expandable balloon 14 mounted on or affixed to a distal end or near the distal end of the catheter 10. The balloon 14 may be designed to be utilized in a variety of medical procedures, including but not limited to a valvuloplasty procedure. One or more elongate members of catheter 10 may extend from a manifold 30 (e.g., manifold 30 shown in FIG. 2) positioned at a proximal end of the catheter 10 to the balloon 14, whereby an inner cavity of the balloon 14 may be in fluid communication with an inflation port of the manifold 30. It can be appreciated that one or more lumens of the catheter 10 may extend through the inner cavity of the balloon 14.

The balloon 14 of the catheter 10 may include a variety of shapes and/or geometries in its inflated configuration. By example, the balloon 14 may assume and conform to the following shapes and geometries in its inflated configuration: hourglass, pear-shaped, tear-drop, circular, oblong, ellipsoid, spherical, intermittently spherical, intermittently oblong, intermittently circular, intermittently ellipsoid, missile-shaped, tapered, a repeating taper, a patterned taper, tubular, intermittently tubular, arcuate, intermittently arcuate or any equivalent or known shape or geometry in the art.

As illustrated in FIG. 2, the catheter 10 may include an outer elongate member 12 (i.e., sheath, outer sheath) and an inner elongate member 40. In at least some examples, the outer elongate member 12 may include a proximal end region, a distal end region and a lumen extending therein. The outer elongate member 12 may include an inner lumen extending from a proximal end of the outer elongate member 12 to a distal end of the outer elongate member 12. The lumen of the outer elongate member 12 may be utilized to transport inflation fluid from the inflation port 32 of the manifold 30 to the balloon 14. In some examples, the outer elongate member 12 may include at least one lumen or at least one or more lumens. The catheter 10 may further include an inner elongate member 40 extending within a portion of the lumen of the outer elongate member 12 and the balloon 14. The distal end region of the inner elongate member 40 may further be coupled to the distal waist 18 of the balloon 14.

In this and other examples, the catheter 10 may further include a manifold 30 coupled to the proximal end region of the outer elongate member 12. The manifold 30 may include lumens, ports, apertures, openings, any combination or permutation of the aforementioned, or any equivalent structure known in the art. Further, the manifold 30 may include a plurality of lumens, ports, apertures, openings, any combination or permutation of the aforementioned, or any equivalent structure known in the art. In this and other examples, the catheter 10 may further include a tensioning member 44. The tensioning member 44 may be coupled to the proximal end region of the inner elongate member 40, and may be configured to apply tension (i.e., tensile force, axial force) to the inner elongate member 40 which, in turn, may transfer axial tension to the balloon 14. The tensioning member 44 may be formed of any suitable and known material, including but not limited to a polymer, a combination of polymers, nylon, a combination of nylons, an alloy, a combination of alloys, a metal, a combination of metals, a plastic, a combination of plastics, a thermoplastic, a combination of thermoplastics or any combination or permutation of the aforementioned materials and any known materials in the art. The tensioning member 44 may be configured to translate (i.e., moving the tensioning member 44 forward, backward, laterally, forward and backward, rotating the tensioning member 44 clockwise, rotating the tensioning member 44 counterclockwise, rotating the tensioning member 44 both clockwise and counter-clockwise or moving by twisting or applying torque) such that when translating the tensioning member 44 relative to the manifold 30 (i.e., moving or actuating the tensioning member 44), the tensioning member 44 may impart and maintain axial tension on the inner elongate member 40, the balloon 14 or both the inner elongate member 40 and the balloon 14 during deflation of the balloon 14.

It can be appreciated that when the tensioning member 44 imparts tension to the inner elongate member 40, the balloon 14 or both the inner elongate member 40 and the balloon 14 during deflation of the balloon 14, no tensile force or axial force applied to the balloon 14 may be lost. In other words, the forces applied to the balloon 14 (e.g., axial, tensile, radial, etc.) may not dissipate from the balloon 14 during deflation. Accordingly, imparted axial tension forces may remain at the same level of tension applied by the tensioning member 44 throughout deflation of balloon 14, such that the balloon 14 deflates into a deflated configuration in which the maximum diameter of deflated balloon 14 may be equal to or less than an outer diameter of outer elongate member 12, thereby allowing tight re-folding and withdrawal of deflated balloon 14 into an introducer sheath utilized to introduce the balloon catheter 10 into a body vessel of a patient, for example.

In some examples, when axial tension is imparted to inner elongate member 40, and/or the balloon 14, the balloon 14 may deflate and re-fold for withdrawal into an introducer sheath whereby a maximum diameter of the deflated balloon may be equal to or less than an outer diameter of the outer elongate member 12. In other words, when the axial tension is imparted maintained on the inner elongate member 40 and/or the balloon 14, the balloon 14 may re-fold upon itself such that the maximum diameter of the deflated balloon 14 is less than or equal to a maximum outer diameter of the outer elongate member 12. In other examples, the balloon 14 may re-fold under tension (as described above), such that the outer diameter of the balloon 14 is greater than the outer diameter of the outer elongate member 12, yet the re-folded diameter of the balloon 14 may be substantially compact, thereby permitting case of withdrawal (e.g., reduced withdrawal forces) of the balloon 14 into an introducer sheath utilized to introduce the balloon 14 into a body vessel of a patient. Additionally, improving refolding (reduced cross-section profile of balloon upon deflation) may increase the tension on the inner elongate member 40 may also help “straighten” the distal end region of the balloon catheter 10, which may further reduce withdrawal forces of the balloon 14 into an introducer sheath.

The balloon 14 may include a balloon wall constructed of one or more layers, wherein each of the layers may be constructed from different balloon materials. For example, the wall of balloon 14 may include an inner layer and an outer layer, whereby the inner layer is constructed of a lower durometer (e.g., softer) material as compared to the outer layer. This two-layer construction may be referred to as a bi-layer balloon base layer. Further, the inner and outer layer of the bi-layer balloon wall of balloon 14 may be co-extruded during the manufacturing process of the balloon 14. Typical balloon materials may include polymer materials, some examples of which are listed herein. Additionally or alternatively, the wall of balloon 14 may include an inner layer and an outer layer, whereby the outer layer is constructed of a lower durometer (e.g., softer) material as compared to the inner layer. Additionally or alternatively, multiple layers are contemplated for construction of the balloon 14. In some examples, the balloon 14 may include one or more layers, two or more layers, three or more layers, four or more layers, five or more layers. The layers of the balloon 14 may have identical durometers, differing durometers, or a combination or permutation of the aforementioned. In yet additional examples, the balloon wall of balloon 14 may be formed as a tri-layer, wherein the outer layer and the inner layer possess a softer durometer than the middle layer. Alternatively or additionally, the balloon wall of balloon 14 may be formed as a tri-layer, wherein the middle layer possesses a softer durometer than both the outer and inner layers. Alternatively or additionally, the layers in a tri-layer configuration may all possess differing durometers, the same durometer, or a scheme of durometers where two layers possess the same durometer and one layer possesses a durometer different than the other two layers. Alternatively or additionally, a greater number of layers may be present within the wall of balloon 14. Balloon 14 may be designed in a quad-layer construction (i.e. having four layers) and each of the layers may possess the same durometer or a different durometer than all other layers. In yet other examples of quad-layer construction, the wall of balloon 14 may have two layers of the same durometer and may also have two different layers of differing durometer.

Alternatively or additionally, the quad-layer construction may feature three layers of the same durometer and a fourth layer possessing a greater or lesser durometer than the other three layers. All possible permutations, combinations and arrangement of layers as is known in the art is further contemplated.

FIG. 2 illustrates that the tensioning member 44 may be coupled to the proximal end region of the inner elongate member 40. A user of the catheter 10 may actuate the tensioning member 44 (i.e., translate the tensioning member 44 axially, horizontally, rotationally, and the like). It can be appreciated that as a user of the device translates the tensioning member 44 in a proximal-to-distal direction (e.g., toward the balloon 14), the tensioning member 44 may impart axial tension on the inner elongate member 40, the balloon 14 or both the inner elongate member 40 and the balloon 14. Additionally, translation of the tensioning member 44 toward the balloon 14 may maintain axial tension on the balloon 14 such that when the balloon 14 is deflated, axial tension may be imparted to the balloon 14 during the re-fold process. The axial tension imparted to the balloon 14 during the re-fold process may encourage the balloon 14 to re-fold to a substantially compact configuration which may reduce the withdrawal force required to withdraw the balloon 14 into an inner lumen of an introducer sheath.

FIG. 2 further illustrates that the catheter 10 may include a manifold 30 having a guidewire port 48. The guidewire port 48 may have a first locking region 42. The tensioning member 44 may include a second locking region 46 that may be configured to engage the first locking region 42 of the manifold 30, or vice-versa. Alternatively or additionally, the first locking region 42 of manifold 30 may interlock with the second locking region 46 of the tensioning member 44, or vice-versa. In yet other examples, the first locking region 42 of the manifold 30 may irreversibly interlock with the second locking region 46 of the tensioning member 44, or vice-versa. In other words, the first locking region 42 of the manifold 30 may interlock with the second locking region 46 of the tensioning member 44 by twisting one or both of the first and second locking regions 42, 46 in a first direction (e.g., clockwise or counterclockwise direction), but may not be unlocked by twisting one or both of the first and second locking regions 42, 46 in a second direction opposite the first direction.

In further examples, the first locking region 42 of the manifold 30 may reversibly interlock with the second locking region 46 of the tensioning member 44, or vice-versa. In other words, the first and second locking regions 42,46 may be interlocked by twisting one or both of the first and second locking regions 42, 46 in a clockwise direction, while the first and second locking regions 42, 46 may be unlocked by twisting one or both of the first and second locking regions 42, 46 in a counterclockwise direction, or vice-versa.

As shown in at least FIG. 2, an inflation port 32 of the manifold 30 may further include threads 34. In some examples, the threads 34 may include internal threads, external threads, or both internal and external threads, all configured to engage a threaded region of an inflation device (e.g., syringe) or the like. In further examples, the manifold 30 may include multiple inflation ports and multiple threaded regions configured for threaded engagement of various components and medical devices. The threads of these and other examples may possess varying pitch, diameter and thread angle. The threaded regions of the manifold may also possess different pitches, thread diameters and thread angles relative to other threaded regions of the manifold or disposed about the catheter 10.

After the desired amount of tension is imparted onto one or more of the balloon 14 and/or the inner elongate member 40, the tensioning member 44 may be locked in position, and therefore impart tension onto the balloon 14 and/or the inner elongate member 40 by engagement of the first locking region 42 of manifold 30 with the second locking region 46 of tensioning member 44. Additionally or alternatively, one or both of the first and second locking regions 42, 46 may include threads, threaded locks, or threaded locking components. In yet other examples one or both of the first and second locking regions 42, 46 may include interference fit or snap-fit components, in which the first and second locking regions may interlock (i.e., lock together, snap together) via an interference fit connection, a snap-fit connection, an annular snap-fit connection, a concentric connection, a press-fit connection or the like. Additionally or alternatively to the above, one or both of the first and second locking regions 42, 46 may include internal threads or external threads and the first and second locking regions 42, 46 may interlock (i.e., locked together) by a screw-fit, a torque-fit, or a twist-fit connection. In yet other examples, one or both of the first and second locking regions 42, 46 may include corresponding male and female components, and the first and second locking regions 42, 46 may be interlocked (i.e., locked together) through a pin-and-hole connection, a mating fit between male and female components, a snap fit between male and female components, or any connection between male and female components known in the art.

Additionally or alternatively, the first and second locking regions 42, 46 may be reversibly interlocked. In other words, the first and second locking regions 42, 46 may be interlocked by twisting one or both of the first and second locking regions 42, 46 in a counterclockwise direction, while the first and second locking regions 42, 46 may be unlocked by twisting one or both of the first and second locking regions 42, 46 in a clockwise direction, or vice-versa. In yet further examples, both the first locking region 42 and the second locking region 46 may include a plurality of threads. The plurality of threads in both the first locking region 48 and the second locking region 46 may possess consistent thread pitch, varying thread pitch, a patterned thread pitch, consistent thread diameter, varying thread diameter, a patterned thread diameter, consistent thread angle, varying thread angle, a patterned thread angle, or any combination or permutation of the aforementioned.

As shown in FIG. 2, the balloon 14 may include a proximal waist 16, a distal waist 18 and a medial region extending therebetween. An outer elongate member 12 may be coupled to the proximal waist 16 of balloon 14. It can be appreciated that the balloon 14 may extend along a central longitudinal axis of the shaft of catheter 10, and may be tightly refolded along, about, around, partially along, partially about, partially around, substantially along, substantially about, and/or substantially around an outer surface of the inner elongate member 40 of the catheter 10 during and throughout deflation and re-folding of the balloon 14.

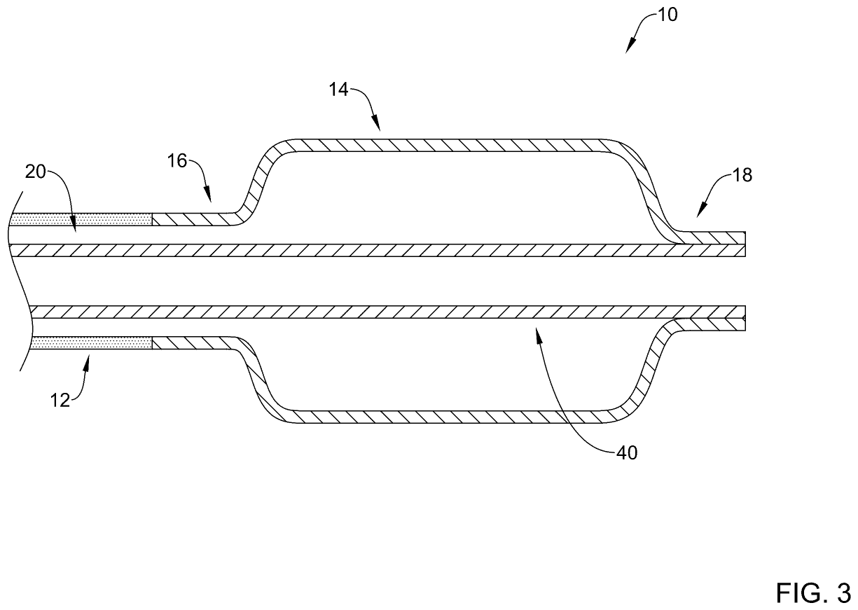

FIG. 3 is a cross-sectional view of the balloon catheter 10 taken along line 3-3 of FIG. 2. As described herein, FIG. 3 illustrates the distal end of the inner elongate member 40 attached to the distal waist 18 of the balloon 14 and the distal end of the outer elongate member 12 attached to the proximal waist 16 of the balloon 14. After deployment into a patient or subject and throughout use of the device, the tensioning member 44 may impart axial tension to the inner elongate member 40 and the balloon 14 to aid the balloon 14 in re-folding into a substantially compact configuration along the outer surface of the inner elongate member 40. As will be described in greater detail herein, the tensioning member 44 may impart and maintain axial tension via various components of the catheter 10 including, but not limited to tubes, springs, tethers, push tubes, compression springs, tension springs, braids, weaves, filaments, rods, push rods, any of the like, any of the equivalent, or any combination or permutation of the aforementioned.

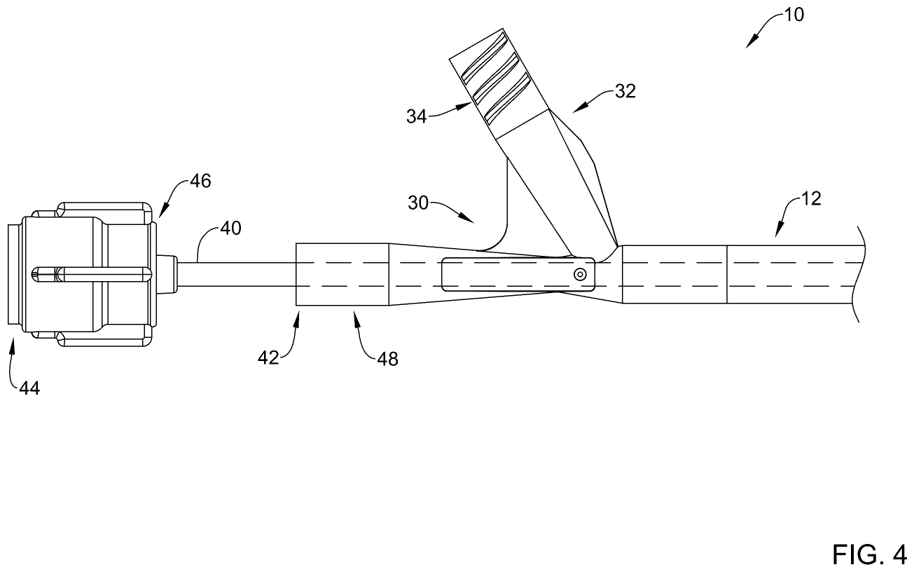

It can be appreciated from FIG. 3 that the inner elongate member 40 may extend within a portion of the inner lumen of the outer elongate member 12. It can be further appreciated that inflation fluid may pass through the inner lumen of the outer elongate member 12 (via the inflation port 32 shown in FIG. 2), such that the inflation fluid flows in an internal, circumferential space 20 extending between an outer surface of the inner elongate member 40 and an inner surface defining the inflation lumen of the outer elongate member 12. In some examples, the diameter of the lumen of the outer elongate member 12 may have a diameter two times greater than the outer diameter of the inner elongate member 40. In some examples, the diameter of the lumen of the outer elongate member 12 may have a diameter that is 1.5 times greater than the outer diameter of the inner elongate member 40, In other words, the diameter of the lumen of the outer elongate member 12 may have a ratio of 1.5 times greater diameter than outer diameter of the inner elongate member 40. In other examples, the diameter of the lumen of the outer elongate member 12 may be 1.1 times greater, 1.2 times greater, 1.3 times greater, 1.4 times greater, 1.6 times greater, 1.8 times greater, 2.1 times greater, 2.3 times greater, 2.5 times greater, 2.75 times greater or 3 or more times greater than the outer diameter of the inner elongate member 40. FIG. 4 depicts a detailed view of a proximal end region of a catheter 10 of the present disclosure. As shown in FIG. 4, a tensioning member 44 may be coupled to the proximal end region of the inner elongate member 40. FIG. 4 further illustrates the tensioning member 44 shown in FIG. 4 is spaced away from the guidewire port 48. The tensioning member 44 of this and other examples may be a sliding mechanism, an axial drive device, an axial sliding mechanism, a torque drive device, a torque drive mechanism, a pneumatic drive device, a pneumatic drive mechanism, an electric drive device, an electric drive mechanism, a rachet drive device, a ratchet drive mechanism, any of the like, any of the equivalent or any combination or premutation of the aforementioned. The tensioning member 44 may include a cap, a knurled handle, a round handle, a blunt handle, a dial, a knob, a projection, a button, a manipulatable member, a projecting handle, a rectangular handle, a lever, or any equivalent handle or leveraging means known in the art.

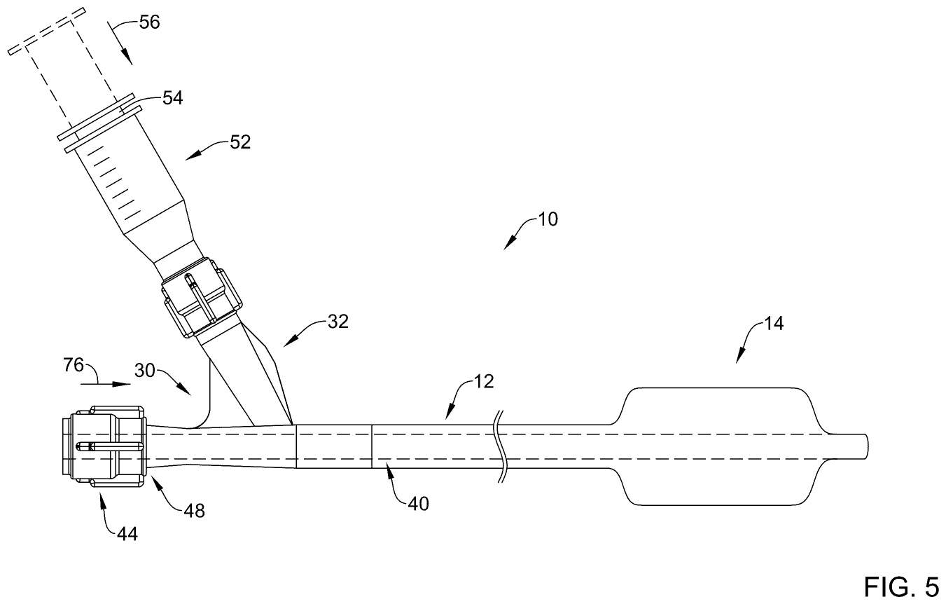

FIG. 5 illustrates actuation of the tensioning member 44 to impart and maintain tension on the inner elongate member 40, the balloon 14 or both the inner elongate member 40 and the balloon 14. The arrow 76 of FIG. 5 illustrates that a user may translate the tensioning member 44 from a first position in which the tensioning member 44 is spaced away from the guidewire port 48 of the manifold 30 (as shown in FIG. 4) to a second position in which the tensioning member 44 is engaged with the guidewire port 48 of the manifold 30 to impart and maintain tension on the inner elongate member 40, the balloon 14 or both the inner elongate member 40 and the balloon 14. For example, the arrow 76 in FIG. 5 illustrates the tensioning member 44 being translated in a proximal-to-distal direction from its first position to a second position, whereby the proximal-to-distal translation may impart axial tension to the inner elongate member 40, the balloon 14 or both the inner elongate member 40 and the balloon 14.

In some instances, the engagement of the tensioning member 44 with the guidewire port 48 of the manifold 30 may be confirmed by tactile, haptic, visual and/or audible feedback. It is also contemplated that improper locking and/or unlocking of the tensioning member 44 to the manifold 30 may be communicated to a user or practitioner by tactile, haptic, visual or audible feedback. Examples of audible feedback may include sounds such as clicking, popping, beeping, snapping or any equivalent sound or sound known in the art. Examples of visual feedback include visual alerts, visual indicators, the appearance of indicia, the illumination of a light, pulsed lighting, strobed lighting, patterned lighting or any equivalent or known visual feedback in the art. Examples of haptic feedback may include vibration, pulses, or any equivalent or known haptic feedback in the art.

It can be appreciated that the manifold 30 described herein may include a plurality of ports, apertures and/or lumens. In an example, the manifold 30 may include an inflation port 32 having an inflation lumen for accepting and transferring inflation media (i.e., saline) to the balloon 14.

FIG. 5 further illustrates that an inflation device 52 (e.g., syringe, pump, etc.) may be attached to the manifold 30 such that it communicates with an inflation lumen of the inflation port 32 of catheter 10. Inflation device 52 may be any pump known in the art, including but not limited to a syringe pump, multiple syringe pumps, an infusion pump, multiple infusion pumps, a pulsatile pump, multiple pulsatile pumps, an electric pump, multiple electric pumps, a diaphragm pump, multiple diaphragm pumps, a gravity-fed pump, multiple gravity-fed pumps, any of the equivalent, any of the like, or any combination or permutation of the aforementioned. The balloon 14 may be inflated by action of an inflation device 52. In this and other examples, inflation device 52 may be a syringe and the balloon 14 may be inflated with inflation fluid by depressing a piston 56 of the inflation device 52 as depicted by the directional arrow 56 shown in FIG. 5.

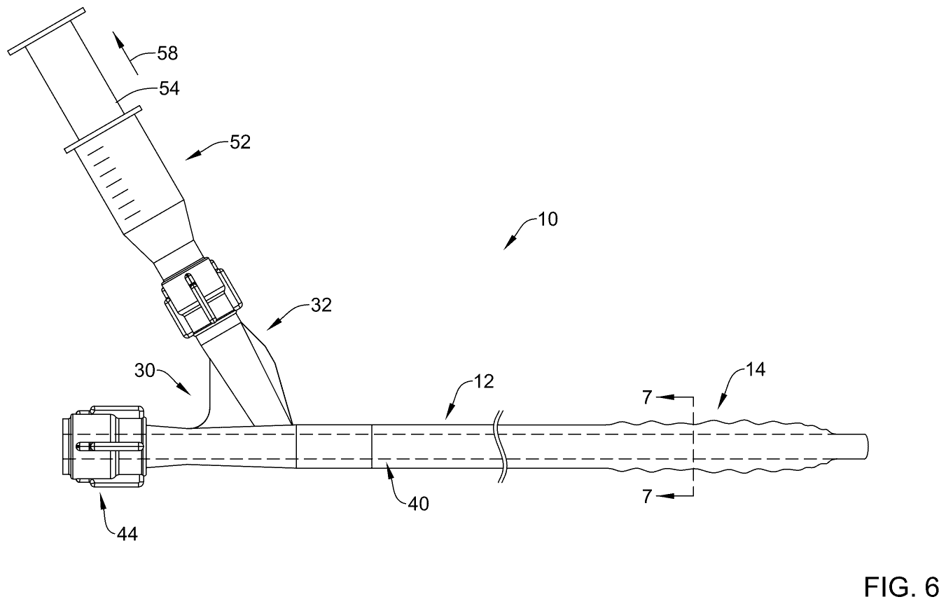

FIG. 6 is a profile view of the catheter 10 shown in FIG. 5 whereby the balloon 14 is shown in a deflated, re-folded state. As shown in FIG. 6, as the balloon begins to re-fold, it may wrap around the outer surface of the inner elongate member 40 of catheter 10. As discussed herein, during re-folding of the balloon 14, the tensioning member 44 may impart axial tension on the inner elongate member 40 and/or the balloon 14. The balloon 14 may be deflated by action of the inflation device 52. In this and other examples, inflation device 52 may be a syringe pump, and the balloon 14 may be deflated by retracting the piston 54 of inflation device 52 as depicted by the directional arrow 58 shown in FIG. 6. As discussed herein, during deflation, axial tension may be maintained on the inner elongate member 40 and/or the balloon 14, whereby the axial tension imparted to the balloon 14 may be released via unlocking, decoupling, or disconnecting the tensioning member 44 from the manifold 30.

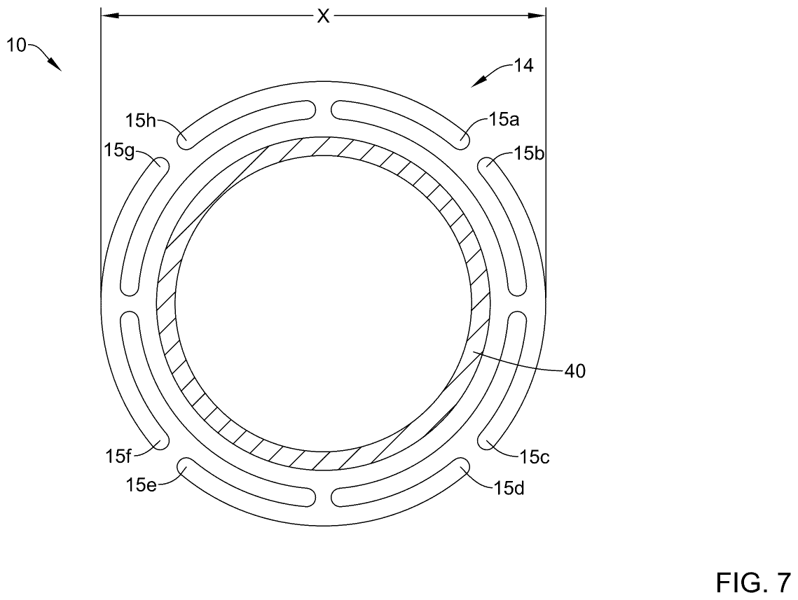

FIG. 7 is a cross-sectional view of the balloon 14 taken along line 7-7 of FIG. 6. FIG. 7 illustrates the balloon 14 in a deflated and re-folded configuration. As discussed herein, axial tension may be maintained on the inner elongate member 40 and/or the balloon 14 as the balloon 14 is deflated. In some examples, such as that illustrated in FIG. 7, the balloon may refold into a configuration in which the balloon 14 forms a plurality of folds 15A-15H arranged around the inner elongate member 40. For example, FIG. 7 illustrates that when the balloon 14 is deflated under tension, it may form eight individual “T-shaped” folds 15A-15H arranged circumferentially around the inner elongate member 40. This is not intended to be limiting. Rather, it is contemplated that when deflated under tension, the balloon 14 may form 1, 2, 3, 4, 5, 6, 7, 8, 9, 10 or more folds arranged around the inner elongate member 40. Additionally, it is contemplated that the balloon may re-fold in a variety of different fold configurations. For example, the balloon 14 may re-fold into a plurality of “wrapped” folds which lay on top of one another. In some examples, the wrapped folds may all point in the same direction around the inner elongate member 40. In other examples, one or more of the wrapped folds may all point in different directions around the inner elongate member 40.

FIG. 7 further illustrates that the re-folded balloon 14 may include an outer diameter “X”. In some examples, the outer diameter X of the re-folded balloon 14 may be about 4.0 mm to about 8.0 mm, or about 4.5 mm to about 7.5 mm, or about 5.0 mm to about 7.0 mm, or about 5.5 mm to about 6.5 mm, or about 6.0 mm. Additionally, the cross-sectional shape of the re-folded balloon 14 shown in FIG. 7 is not intended to be limiting. Rather, the balloon 14 may re-fold into a variety of cross-sectional shaped folds and/or configurations while maintaining an outer diameter of 6.0 mm or less. In some examples, the one or more of the folds 15A-15H of the balloon 14 may overlap each other while still maintaining an outer diameter of 6.0 mm or less. Further, one or more of the folds 15A-15H may be longer or shorter than one or more of the folds 15A-15H while still maintaining an outer diameter of 6.0 mm or less.

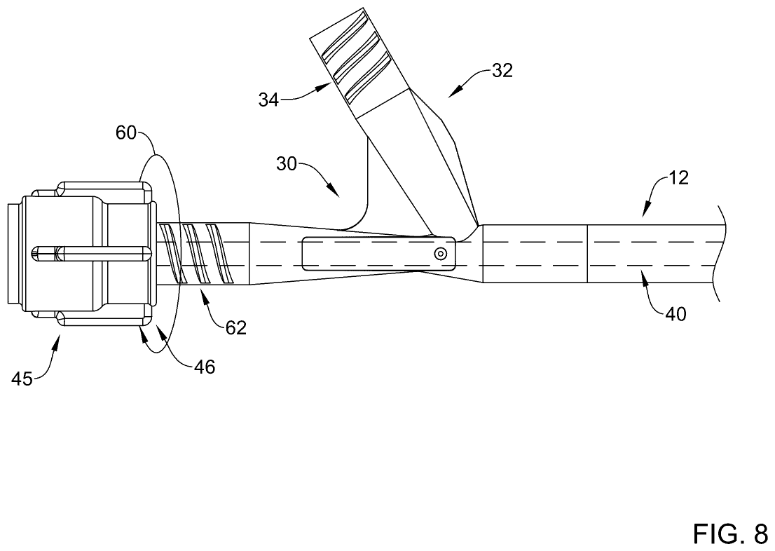

FIG. 8 shows a detailed view of a proximal end region of another example catheter 10 of the present disclosure. In the example shown in FIG. 8, a tensioning member 45 may be coupled to the proximal end region of the inner elongate member 40. FIG. 8 illustrates that the manifold 30 may include a threaded region 62 configured to engage an internal threaded region of the tensioning member 45. It can be appreciated that the tensioning member 45 may be configured to rotate (illustrated by the arrow 60) relative to the manifold 30. It can be further appreciated that rotation (e.g., clockwise rotation as illustrated by the arrow 60) of the tensioning member 45 relative to the manifold 30 may translate the tensioning member 45 along the manifold 30 in a proximal-to-distal direction relative to both the manifold 30 and the balloon 14. Accordingly, rotation of the tensioning member 45 relative to the manifold 30 may impart tension (e.g., axial tension) on the inner elongate member 40, the balloon 14 or both the inner elongate member 40 and the balloon 14. As discussed herein, by applying and maintaining tension on the inner elongate member 40 and/or the balloon 14, withdrawal forces of the balloon 14 through an introducer sheath may be reduced as the balloon 14, while under maintained tension, may tightly re-fold about the outer surface of the inner elongate member 40.

It can be further appreciated that rotating the tensioning member 45 in a counterclockwise direction may reduce tension on the inner elongate member 40, the balloon 14 or both the inner elongate member 40 and the balloon 14. It can be further appreciated that the example catheter 10 shown in FIG. 8 may be configured such that tension may be increased through counterclockwise rotation of the tensioning member 45 and tension may be decreased through clockwise rotation of the tensioning member 45.

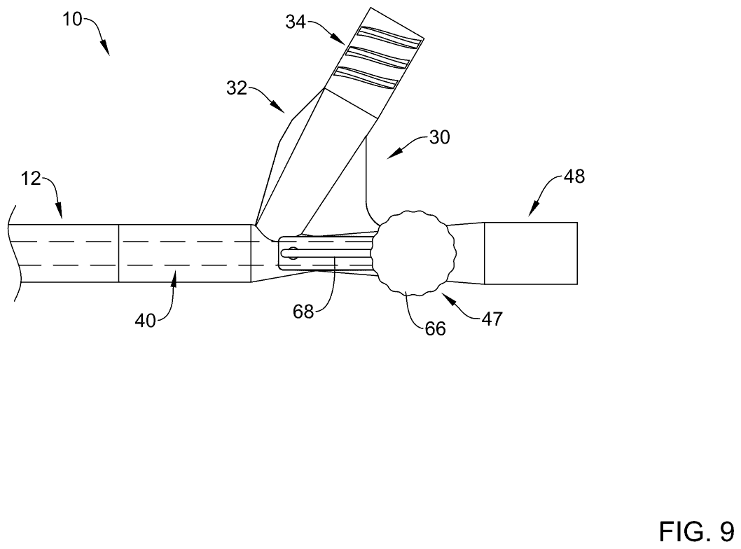

FIG. 9 shows a detailed view of a proximal end region of another example catheter 10 of the present disclosure. Similar to the example discussed with respect to FIG. 2, the inflation port 32 of the manifold 30 may include threads 34. In some examples, the threads 34 may include internal threads, external threads, or both internal and external threads, all configured to engage a threaded region of an inflation device (e.g., syringe) or the like. In other examples, the manifold 30 may include multiple inflation ports and multiple threaded regions configured for threaded engagement of various components and medical devices. The threads of these and other examples may vary in pitch, diameter and thread angle. The threaded regions of the manifold 30 may also possess different pitches, thread diameters and thread angles relative to other threaded regions of the manifold or disposed about the catheter 10. Further, threads of this and any other examples may be any type of threading or threaded connection known in the art, including but not limited to right-handed threads, left-handed threads, helical threads, intermittent threads, any of the like, any of the equivalent or any combination or permutation of the aforementioned.

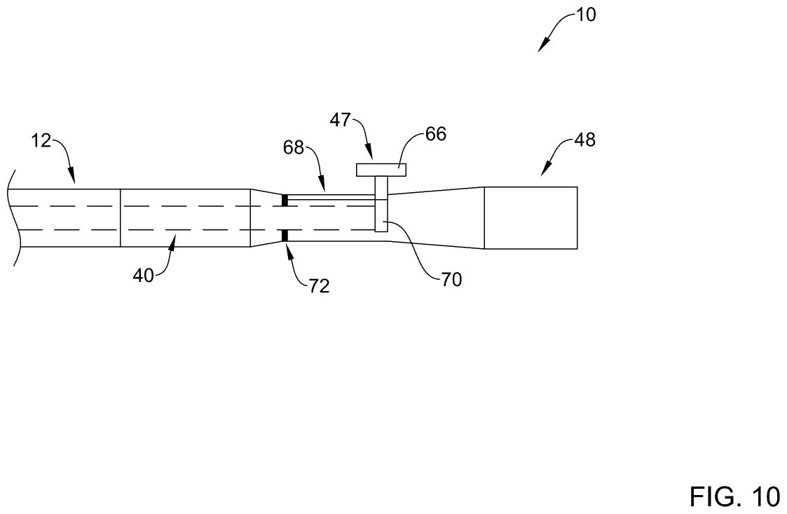

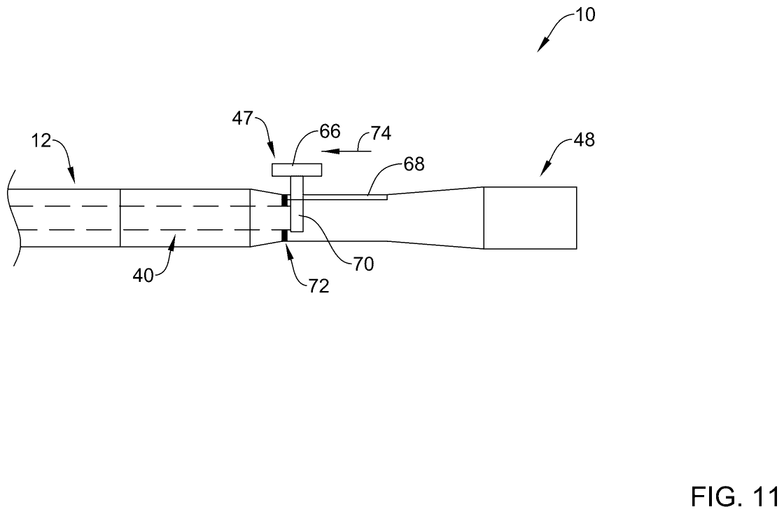

As shown in FIG. 9, the catheter 10 may include a tensioning member 47 coupled to the manifold 30. Further, the tensioning member 47 may include an actuation handle 66. The actuation handle 66 may include a knob, dial, or other rotatable and/or manipulatable object. Additionally, as will be described in greater detail with respect to FIGS. 10-11, a portion of the tensioning member 47 may extend through a longitudinal slot 68 (e.g., aperture) positioned through a wall of the manifold 30. Further, the portion of the tensioning member 47 extending through the slot 68 in the manifold 30 may be coupled to the distal end of the inner elongate member 40 positioned within the manifold 30. It can be appreciated that the manifold 30 may further include an oversized captive O-ring configured to prevent fluid from leaking through the slot 68.

FIG. 10 illustrates the catheter shown in FIG. 9 whereby the manifold 30 has been rotated 90 degrees into the page. Accordingly, it can be appreciated that the inflation port 32 shown in FIG. 9 is hidden from view in FIG. 10.

As discussed herein, FIG. 10 illustrates that the tensioning member 47 may include a stem 70 extending through the slot 68. Further, FIG. 10 illustrates that a portion of the stem 70 may be coupled to the proximal end of the inner elongate member 40. It can be appreciated that the stem 70 may be configured to permit a guidewire to extend therethrough and into a lumen of the inner elongate member 40. For example, the stem 70 may include an aperture which is aligned with the lumen of the inner elongate member. Accordingly, a guidewire may be advanced through a guidewire port 48 of the manifold 30, through the aperture positioned in the stem 70 and into the lumen of the inner elongate member 40.

FIG. 10 further illustrates that the catheter 10 may further include a lumen seal 72 positioned distal to the slot 68. Further, FIG. 10 illustrates that the inner elongate member 40 may extend through an aperture in the lumen seal 72. The lumen seal 72 may be configured to prevent inflation media from leaking out of the guidewire port 48 of the manifold 30. In other words, the lumen seal 72 may be configured to provide a seal between an inner surface of the manifold 30 and the outer surface of the inner elongate member 40, thereby preventing inflation media injected through the inflation port 32 (shown in FIG. 9) from leaking out of the guidewire port 48 of the manifold 30.

Lumen seal 72 may be any seal, valve, scaling component or valve component known in the art. For example, lumen seal 72 may be a gasket, an O-ring, a split-ring, a partial gasket, a partial O-ring, an annular valve, a duckbill valve, a ball valve, a flapper valve, a pressure valve, or any combination of the aforementioned. As shown in FIG. 10, but not limited to this example, lumen seal 72 may be an annular O-ring placed proximal to the inflation port 32 of the manifold 30, such that inflation media may flow through inflation port 32 and into the outer elongate member 12 without leaking out of the guidewire port 48. Additionally, any number and/or combination of seals, valves and their position within or along catheter 10 is contemplated.

FIG. 10 further illustrates the actuation handle 66 which may be configured to permit a user to actuate (e.g., translate, slide) the tensioning member 47 within the slot 68 in a proximal-to-distal direct to impart axial tension onto inner elongate member 40, the balloon 14 or both the inner elongate member 40 and the balloon 14.

FIG. 11 illustrates the catheter 10 illustrated in FIG. 10 whereby the tensioning member 47 has been advanced in a proximal-to-distal direction within the slot 68. As discussed herein, the proximal end of the inner elongate member 40 may be coupled to the tensioning member 47. FIG. 11 further illustrates the lumen seal 72 as described herein. As shown in FIGS. 10-11 and described herein, after the balloon 14 has been inflated, tension may be applied to the inner elongate member 40, the balloon 14 or both the inner elongate member 40 and the balloon 14 during deflation and re-wrap of the balloon 14 prior to withdrawing the balloon 14 from the patient through an introducer sheath. It can be appreciated that to impart tension to the inner elongate member 40, the balloon 14 or both the inner elongate member 40 and the balloon 14 during deflation and re-wrap of the balloon 14, a user may grasp the actuation handle 66 of the tensioning member 47 and translate the tensioning member 47 in a proximal-to-distal direction (the proximal-to-distal translation of the tensioning member 47 is indicated by the arrow 74 in FIG. 11), thereby applying axial tension to the tensioning member 47 which may impart this tension to the inner elongate member 40, the balloon 14 or both the inner elongate member 40 and the balloon 14.

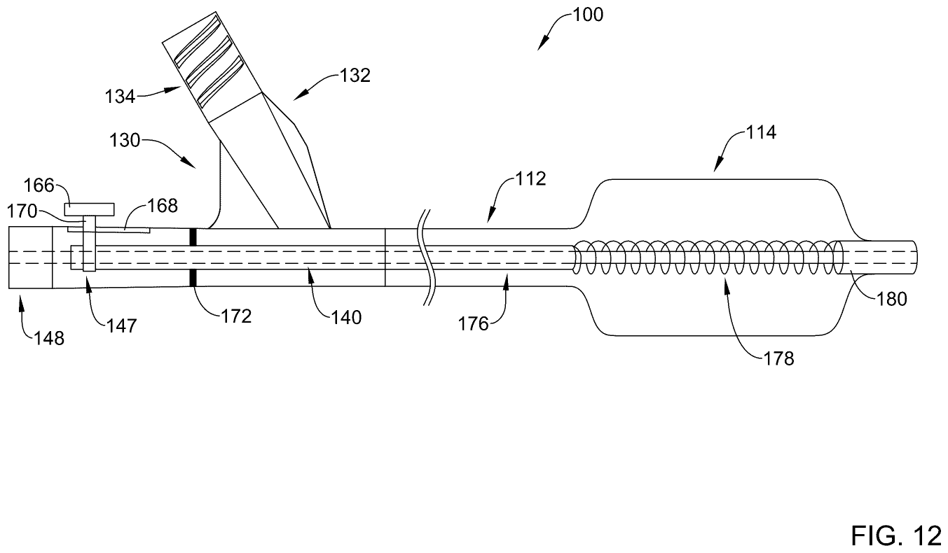

FIG. 12 illustrates another example catheter 100 of the present disclosure. Similar to other catheters described herein, the catheter 100 may include an outer elongate member 112 having a distal end coupled to the proximal end region of a balloon 114 and a proximal end coupled to a manifold 130. The catheter 100 may further include an inner elongate member 140 having a proximal end coupled to the manifold 130 (e.g., attached to a portion of the guidewire port 148 of the manifold 130) and a distal end coupled to a distal balloon sleeve 180. In some examples, the distal balloon sleeve 180 may be coupled to a distal end region of the balloon 114. The inner elongate member 140 may extend through the guide wire port 148 of the manifold 130, through a lumen of the outer elongate member 112 and through the inner cavity of the balloon 114. Similar to other catheters described herein, the manifold 130 may include an inflation port 132 having threads 134. The threads 134 may include internal, external threads, or both internal and external threads, all configured to engage a threaded region of an inflation device (e.g., syringe) or the like. In other examples, the manifold 130 may include multiple inflation ports and multiple threaded regions configured for threaded engagement of various components and medical devices. FIG. 12 further illustrates that the catheter 100 may include a tensioning member 147 having a stem 170 extending through a slot 168 in the wall of the manifold 130, similar to the example catheter disclosed with respect to FIGS. 9-11. Additionally, the tensioning member 147 may include an actuation handle 166 attached to the stem 170. Further, the stem 170 may be coupled to the proximal end region of a proximal push tube 176 disposed over the inner elongate member 140 (e.g., the inner elongate member 140 may extend through a lumen of the proximal push tube 176). Further, the proximal push tube 176 may extend within a portion of both the manifold 130 and the lumen of the outer tubular member 112, whereby a distal end of the proximal push tube 176 may engage a compression spring 178 disposed over the inner elongate member 140 (e.g., the inner elongate member 140 may extend through the compression spring 178). In some examples, the compression spring 178 may be positioned within a cavity of the balloon 114. Further, FIG. 12 illustrates that the compression spring 178 may extend through the inner cavity of the balloon 114 whereby a distal end of the compression spring 178 may engage a proximal end of the distal balloon sleeve 180. As illustrated in FIG. 12, the inner elongate member 140 may extend through the proximal push tube 176, the compression spring 178 and the distal balloon sleeve 180, whereby the proximal push tube 176, the compression spring 178 and the distal balloon sleeve 180 are all axially aligned with one another. As described herein, the distal end of the inner elongate member 140 may be attached to the distal balloon sleeve 180.

The push tube 176 may be formed of any known material in the art, including but not limited to an alloy, a combination of alloys, a metal, a combination of metals, a plastic, a combination of plastics, a polymer, a combination of polymers, a thermoplastic, a combination of thermoplastics, or any combination or permutation of these aforementioned materials or any known or equivalent material in the art.

In some examples, the catheter 100 may include one or more compression springs and push tubes disposed along the inner elongate member 140, in an alternating fashion. In other words, along the length of the inner elongate member 140, a first compression spring may be disposed, followed by a push tube disposed distally of the first compression spring, followed by a second compression spring distal of the push tube and a second push tube disposed distally of the second compression spring. Any known pattern is contemplated. In other words, a pattern of two compression springs followed by a single push tube is contemplated. In this and other examples, the push tube 176 may be positioned between two or more compression springs, proximal to the compression spring 176 or distal to the compression spring 176. In yet other examples, the catheter 100 may include only push tubes. The push tubes may be of a monolithic construction, pieced together along and disposed over the inner elongate member 140, or in an intermittent pattern along the inner elongate member 140.

Further, in some examples, the catheter 100 may include three or more compression springs disposed over the inner elongate member 140. Alternatively or additionally, all compression springs may be adjacent to one another. In yet further examples, each compression spring may be spaced apart from each other by an equal distance. In other examples, each compression spring may be spaced apart from each other by different or varying distances.

Further, in some examples, the catheter 100 may include three or more push tubes disposed over the inner elongate member 140. Alternatively or additionally, all push tubes may be adjacent to one another. In yet further examples, each push tube may be spaced apart from each other by an equal distance. In other examples, each push tube may be spaced apart from each other by different or varying distances. The compression springs of this and other examples may alternatively be substituted with other springs including, but are not limited to, tension springs, extension springs, torsion springs, spiral springs or the like.

After deployment into a patient or subject and throughout use of the device, the tensioning member 147 may be utilized to impart axial tension to the inner elongate member 140 and/or the balloon 114 to aid the balloon in re-folding to a substantially compact configuration along the outer surface of the inner elongate member 140. For example, a user may grasp the actuation handle 166 of the tensioning member 147 and translate the tensioning member 147 within the slot 168 in a distal-to-proximal direction. It can be appreciated that translating the tensioning member 147 within the slot 168 in a distal-to-proximal direction may impart axial tension to the inner elongate member 140, the balloon 114 or both the inner elongate member 140 and the balloon 114 214 via a force transmission through the proximal push tube 176 and the compression spring 178.

In some examples, after the desired tension within all desired components is achieved, the actuation handle 166 may be actuated to lock the tensioning member 147 in place relative to the manifold 130. In some examples, the actuation handle 166 may be configured to include a threaded region configured to engaged a threaded region of the stem 170. Accordingly, in some examples, the actuation handle 166 may be configured to rotate and translate along the stem 170, whereby the actuation handle 166 may be further configured to lock the tensioning member 147 in place relative to the manifold 130.

After the tensioning member is locked in place, the balloon 114 may be deflated while tension is maintained on the inner elongate member 140, the balloon 114 or both the inner elongate member 140 and the balloon 114. As the balloon 114 deflates under tension, the balloon 114 may tightly re-fold about the outer surface of the inner elongate member 140. After the balloon 114 re-folds about the outer surface of the inner elongate member 140, the balloon 114 may be withdrawn through an introducer sheath and out of the patient.

In some examples, the actuation handle 166 may also include a button or a lever. Further, actuation of the actuation member may include pressing a button, rotating a button, pressing a lever, pushing a lever, rotating a lever, translating a lever or any similar mechanism which may be utilized to lock the tensioning member 147 to the manifold 130.

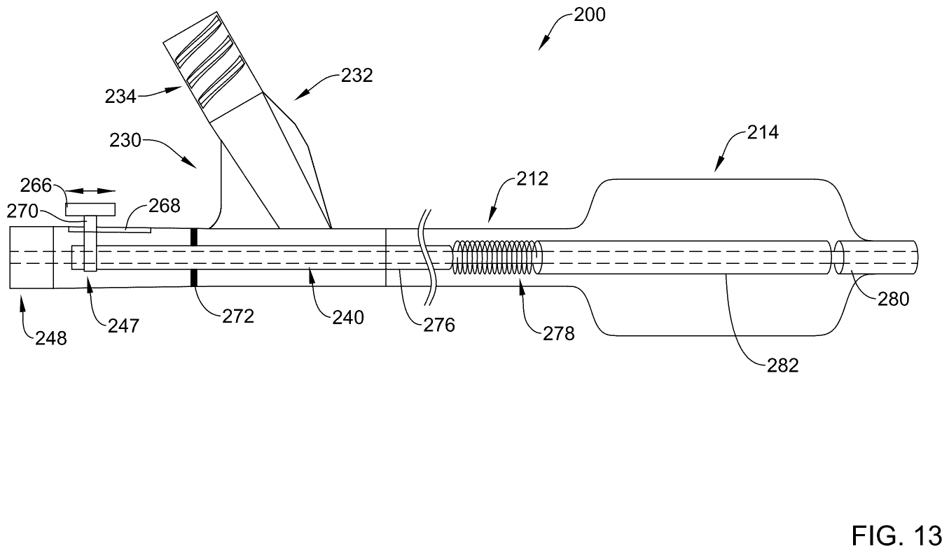

FIG. 13 illustrates another example catheter 200 of the present disclosure. Similar to other catheters described herein, the catheter 200 may include an outer elongate member 112 having a distal end coupled to the proximal end region of a balloon 214 and a proximal end coupled to a manifold 230. The catheter 200 may further include an inner elongate member 240 having a proximal end coupled to the manifold 230 (e.g., attached to a portion of the guidewire port 248 of the manifold 230) and a distal end coupled to a distal balloon sleeve 280. In some examples, the distal balloon sleeve 280 may be coupled to a distal end region of the balloon 214. The inner elongate member 240 may extend through the guide wire port 248 of the manifold 230, through a lumen of the outer elongate member 212 and through the inner cavity of the balloon 214. Similar to other catheters described herein, the manifold 230 may include an inflation port 232 having threads 234. The threads 234 may include internal, external threads, or both internal and external threads, all configured to engage a threaded region of an inflation device (e.g., syringe) or the like. In other examples, the manifold 230 may include multiple inflation ports and multiple threaded regions configured for threaded engagement of various components and medical devices.

FIG. 13 further illustrates that the catheter 200 may include a tensioning member 247 having a stem 270 extending through a slot 268 in the wall of the manifold 230, similar to the example catheter disclosed with respect to FIGS. 9-11. Additionally, the tensioning member 247 may include an actuation handle 266 attached to the stem 270. Further, the stem 270 may be coupled to the proximal end region of a proximal push tube 276 disposed over the inner elongate member 240 (e.g., the inner elongate member 240 may extend through a lumen of the proximal push tube 276). Further, the proximal push tube 276 may extend within a portion of both the manifold 230 and the lumen of the outer tubular member 212, whereby a distal end of the proximal push tube 276 may engage a compression spring 278 disposed over the inner elongate member 240 (e.g., the inner elongate member 240 may extend through the compression spring 278). Further, FIG. 13 illustrates that the catheter 200 may further include a distal push tube 282 positioned distal to the compression spring 278, whereby a distal end of the compression spring 278 may engage a proximal end of the distal push tube 282. Further, FIG. 13 illustrates that a distal end of the distal push tube 282 may engage the proximal end of the distal balloon sleeve 280. The distal push tube 282 may be positioned within a portion of the balloon cavity. As illustrated in FIG. 13, the inner elongate member 240 may extend through the proximal push tube 276, the compression spring 278, the distal push tube 282 and the distal balloon sleeve 280, whereby the proximal push tube 276, the compression spring 278, the distal push tube 282 and the distal balloon sleeve 280 are all axially aligned with one another. As described herein, the distal end of the inner elongate member 240 may be attached to the distal balloon sleeve 280.

The proximal push tube 276 and/or the distal push tube 282 may be formed of any known material in the art, including but not limited to an alloy, a combination of alloys, a metal, a combination of metals, a plastic, a combination of plastics, a polymer, a combination of polymers, a thermoplastic, a combination of thermoplastics, or any combination or permutation of these aforementioned materials or any known or equivalent material in the art.

After deployment into a patient or subject and throughout use of the device, the tensioning member 247 may be utilized to impart axial tension to the inner elongate member 240 and/or the balloon 214 to aid the balloon 214 in re-folding to a substantially compact configuration along the outer surface of the inner elongate member 240. For example, a user may grasp the actuation handle 266 of the tensioning member 247 and translate the tensioning member 247 within the slot 268 in a distal-to-proximal direction. It can be appreciated that translating the tensioning member 247 within the slot 268 in a distal-to-proximal direction may impart axial tension to the inner elongate member 240, the balloon 214 or both the inner elongate member 240 and the balloon 214 via a force transmission through the proximal push tube 276, the compression spring 278 and the distal push tube 282.

In some examples, after the desired tension within all desired components is achieved, the actuation handle 266 may be actuated to lock the tensioning member 247 in place relative to the manifold 230. In some examples, the actuation handle 266 may be configured to include a threaded region configured to engaged a threaded region of the stem 270. Accordingly, in some examples, the actuation handle 266 may be configured to rotate and translate along the stem 270, whereby the actuation handle 266 may be further configured to lock the tensioning member 247 in place relative to the manifold 230.

After the tensioning member is locked in place, the balloon 214 may be deflated while tension is maintained on the inner elongate member 240, the balloon 214 or both the inner elongate member 240 and the balloon 214. As the balloon 214 deflates under tension, the balloon 214 may tightly re-fold about the outer surface of the inner elongate member 240. After the balloon 214 re-folds about the outer surface of the inner elongate member 240, the balloon 214 may be withdrawn through an introducer sheath and out of the patient.

In some examples, one or more components of the catheter examples disclosed herein may be formed from a high strength fiber (e.g., Kevlar® fiber, Aramid® fiber, Pebax® fiber), wire, silk, nylon, or other suitable material. In yet other examples, one or more components of the catheter examples disclosed herein may include an elastic member. In further examples, one or more components of the catheter examples disclosed herein may include a plurality of elastic members. In yet other examples, one or more components of the catheter examples disclosed herein may include an inelastic member or a plurality of inelastic members. In further examples, one or more components of the catheter examples disclosed herein may include both elastic and inelastic members or a plurality of both elastic and inelastic members. One or more components of the catheter examples disclosed herein may also be contemplated as a monolithic or unitary construction and formed of just one material. In other examples, one or more components of the catheter examples disclosed herein may be formed of two or more materials. In yet other examples one or more components of the catheter examples disclosed herein may be formed of three or more materials, four or more materials or five or more materials.

In other examples, the one or more components of the catheter examples disclosed herein may be formed by just one material. In yet other examples, one or more components of the catheter examples disclosed herein may be formed by two or more materials, three or more materials, four or more materials or five or more materials.

In further examples, one or more components of the catheter examples disclosed herein may be formed by just one material. In yet other examples, one or more components of the catheter examples disclosed herein may be formed by two or more materials, three or more materials, four or more materials or five or more materials.

In yet other examples, one or more components of the catheter examples disclosed herein may be formed by just one material. In yet other examples, one or more components of the catheter examples disclosed herein may be formed by two or more materials, three or more materials, four or more materials or five or more materials.

In other examples one or more components of the catheter examples disclosed herein may be formed by just one material. In yet other examples, one or more components of the catheter examples disclosed herein may be formed by two or more materials, three or more materials, four or more materials or five or more materials.

Example polymers utilized to manufacture one or more components of the example balloon catheters disclosed herein may include polymers such as polyethylene terephthalate (PET), polyetherimide (PEI), polyethylene (PE), etc. Some other examples of suitable polymers, including lubricious polymers, may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, a polyether-ester elastomer such as ARNITEL® available from DSM Engineering Plastics), polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example, available under the trade name PEBAX®), silicones, Marlex® high-density polyethylene, Marlex® low-density polyethylene, linear low density polyethylene (for example, REXELL®), polyetheretherketone (PEEK), polyimide (PI), polyphenylene sulfide (PPS), polyphenylene oxide (PPO), polysulfone, nylon, perfluoro (propyl vinyl ether) (PFA), other suitable materials, or mixtures, combinations, copolymers thereof, polymer/metal composites, and the like. In some examples, it may be desirable to use high modulus or generally stiffer materials so as to reduce balloon elongation. The above list of materials includes some examples of higher modulus materials. Some other examples of stiffer materials include polymers blended with liquid crystal polymer (LCP) as well as the materials listed above. For example, the mixture can contain up to about 5% LCP.

It should be understood that this disclosure is, in many respects, only illustrative. Changes may be made in details, particularly in matters of shape, size, and arrangement of steps without exceeding the scope of the disclosure. The disclosure's scope is, of course, defined in the language in which the appended claims are expressed.

What is claimed is:

1. A catheter comprising:
a balloon having a proximal waist, a distal waist and a body portion extending therebetween;
an outer elongate member having a proximal end region, a distal end region and a lumen extending therein, wherein the distal end region is coupled to the proximal waist of the balloon;
an inner elongate member extending within a portion of the lumen of the outer elongate member and the body of the balloon, wherein a distal end region of the inner elongate member is coupled to the distal waist of the balloon;
a manifold coupled to the proximal end region of the outer elongate member; and
a tensioning member coupled to the proximal end region of the inner elongate member;
wherein translating the tensioning member relative to the manifold is configured to impart axial tension on the inner elongate member, the balloon or both the inner elongate member and the balloon during deflation of the balloon.

2. The catheter of claim 1, wherein the manifold includes a first locking region and the tensioning member includes a second locking region configured to engage the first locking region.

3. The catheter of claim 2, wherein engagement of the first locking region with the second locking region is configured to maintain axial tension on the inner elongate member, the balloon or both the inner elongate member and the balloon during deflation of the balloon.

4. The catheter of claim 3, wherein the first locking region is configured to interlock with the second locking region.

5. The catheter of claim 4, wherein the first locking region is configured to irreversibly interlock with the second locking region.

6. The catheter of claim 4, wherein the first locking region is configured to engage the second locking region via an annular snap-fit connection.

7. The catheter of claim 4, wherein interlocking the first locking region with the second locking region provides tactile feedback, audible feedback or both tactile and audible feedback to a user.

8. The catheter of claim 4, wherein the first locking region is configured to reversibly interlock with the second locking region.

9. The catheter of claim 8, wherein the first locking region includes a first plurality of threads and wherein the second locking region includes a second plurality of threads configured to threadedly engage the first plurality of threads.

10. The catheter of claim 1, wherein the tensioning member is spaced away from the manifold in a first configuration, and wherein the tensioning member is translated toward the manifold to impart axial tension on the inner elongate member, the balloon or both the inner elongate member and the balloon.

11. The catheter of claim 1, further comprising an aperture extending along a lateral side of the manifold, and wherein at least a portion of the tensioning member is configured to extend through the aperture, and wherein translating the tensioning member relative to the manifold includes translating the tensioning member along the aperture to impart axial tension on the inner elongate member, the balloon or both the inner elongate member and the balloon.

12. The catheter of claim 1, wherein translating the tensioning member relative to the manifold is configured to impart an axial tension force on the inner elongate member in a range from 1 to 10 Newtons (N).

13. A catheter comprising:
a balloon having a proximal waist, a distal waist and a body portion extending therebetween;
an outer elongate member having a proximal end region, a distal end region and a lumen extending therein, wherein the distal end region is coupled to the proximal waist of the balloon;
an inner elongate member extending within a portion of the lumen of the outer elongate member and the body of the balloon, wherein a distal end region of the inner elongate member is coupled to the distal waist of the balloon;
a manifold coupled to the proximal end region of the outer elongate member;
a tensioning member coupled to the proximal end region of the inner elongate member;
wherein the tensioning member is spaced away from the manifold in a first configuration, wherein the tensioning member is configured to interlock with a portion of the manifold in a second configuration, and wherein translating the tensioning member from the first configuration to the second configuration imparts axial tension on the inner elongate member, the balloon or both the inner elongate member and the balloon during deflation of the balloon.

14. The catheter of claim 13, wherein the tensioning member is configured to irreversibly interlock with the manifold.

15. The catheter of claim 13, wherein the tensioning member is configured to interlock with the manifold via an annular snap-fit connection.

16. The catheter of claim 13, wherein interlocking the tensioning member with the manifold provides tactile feedback, audible feedback or both tactile and audible feedback to a user.

17. The catheter of claim 13, wherein the tensioning member is configured to reversibly interlock with the manifold.

18. The catheter of claim 17, wherein a portion of the tensioning member includes a first plurality of threads and wherein a portion of the manifold includes a second plurality of threads configured to threadedly engage the first plurality of threads.

19. The catheter of claim 13, wherein translating the tensioning member from the first configuration to the second configuration imparts an axial tension force on the inner elongate member in a range from 1 to 10 Newtons (N).

20. A method for treating a body vessel of a patient with a balloon catheter, the method comprising:
advancing a catheter into a body vessel of a patient, the catheter comprising:
a balloon having a proximal waist, a distal waist and a body portion extending therebetween;
an outer elongate member having a proximal end region, a distal end region and a lumen extending therein, wherein the distal end region is coupled to the proximal waist of the balloon;
an inner elongate member extending within a portion of the lumen of the outer elongate member and the body of the balloon, wherein a distal end region of the inner elongate member is coupled to the distal waist of the balloon;
a manifold coupled to the proximal end region of the outer elongate member; and
a tensioning member coupled to the proximal end region of the inner elongate member;
translating the tensioning member relative to the manifold to impart axial tension on the inner elongate member and balloon;
inflating the balloon within the vessel of the patient; and
deflating the balloon while maintaining axial tension on the inner elongate member and balloon.
